(19) United States
(12) Patent Application Publication
(10) Pub. No.: US 2022/0047834 A1
(43) Pub. Date: Feb. 17, 2022

(54) LOWER FACE OXYGEN MASK

(71) Applicant: David Baum, New Port Richey, FL (US)

(72) Inventor: David Baum, New Port Richey, FL (US)

(21) Appl. No.: 16/947,668

(22) Filed: Aug. 12, 2020

(51) Int. Cl. A61M 16/06; A61M 16/00

(52) U.S. Cl. CPC A61M 16/0605; A61M 16/0078; A61M 2202/0208; A61M 16/0683; A61M 16/0672

(57) ABSTRACT

A lower face oxygen mask dimensioned and adapted to circumscribe only the mouth of the wearer is provided. The lower face oxygen mask has spaced apart nostril prongs fluidly connecting the nostrils of the wearer with the spaced enclosed by the body of the lower face oxygen mask. The lower face oxygen mask provides an access aperture adjacent with the mouth of the wearer, the access aperture being compatible with standard size bag valve masks and anesthesia circuits. The body of the lower face oxygen mask also provides an access port for inlet and outlet conduits for oxygen and carbon dioxide flow.

### BACKGROUND OF THE INVENTION

The present invention relates to face masks and, more particularly, a lower face oxygen mask.

Endoscopy procedure uses an endoscope to examine the interior of a hollow organ or cavity of the body. Unlike many other medical imaging techniques, endoscopes are inserted directly into the organ. During endoscopic medical procedures, however, oral access can be problematic; specifically, existing devices do not have an endoscopic scope access port that connects to rescue equipment. This does not work well because during an emergency the existing mask needs to be unattached then removed before access by a rescue device is possible. Furthermore, current devices also cover the lower ⅔ of the face and so invite an improper fit on some patients. The fitness of current masks is challenged by either having to span across the bridge of the nose of the wearer or be sized to circumscribe the eyes of the wearer in addition to the mouth and nose. As a corollary, such sized face masks increase the risk of injury to the eyes and sometimes are not tolerated by patients with claustrophobia.

As can be seen, there is a need for a lower face oxygen mask that solves the problem of oral access during endoscopic medical procedures. The present invention solves this problem by having an access aperture positioned so an endoscopic scope access port can easily access the mouth facilitating a connection to safety equipment for fast emergency rescue without having to remove the mask from the patient. The size of the aperture being the same as and thus compatible with the common rescue devices mask attachment. The present invention also serves the dual purposes of maintaining oxygenation with a higher oxygen flow and providing ventilation for making procedures with sedation safer.

Furthermore, the lower face oxygen mask embodied in the present invention only covers the lower third of the face, improving the air seal with the wearer and increasing the likelihood of being tolerated by people with claustrophobia. Moreover, the prevent invention prevents eye injury by being situated in the nostrils and over the chin.

### SUMMARY OF THE INVENTION

In one aspect of the present invention, a lower face oxygen mask includes the following: a body defined by a periphery; the periphery dimensioned and adapted to circumscribe a mouth and a chin of a wearer and be below a nose of the wearer; and two nostril ports extending upwardly from the periphery for fluidly engages nostrils, respectively, of the wearer and a spaced circumscribed by the body.

In another aspect of the present invention, the lower face oxygen mask includes the following: a body defined by a periphery; the periphery dimensioned and adapted to circumscribe a mouth and a chin of a wearer; two nostril ports extending upwardly from the periphery for fluidly engages nostrils, respectively, of the wearer and a spaced circumscribed by the body; an access aperture in the body, the access aperture adjacent said mouth, wherein the access aperture sized to be compatible with bag valve masks and anesthesia circuits; and an access port for an inlet conduit and an outlet conduit.

These and other features, aspects and advantages of the present invention will become better understood with reference to the following drawings, description and claims.

### BRIEF DESCRIPTION OF THE DRAWINGS

FIG. 1 is a front elevation view of an exemplary embodiment of the present invention; and

FIG. 2 is a side elevation view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a lower face oxygen mask dimensioned and adapted to circumscribe only the mouth of the wearer and provide spaced apart nostril prongs fluidly connecting the nostrils of the wearer with the spaced enclosed by the body of the lower face oxygen mask. The lower face oxygen mask provides an access aperture adjacent with the mouth of the wearer, the access aperture being compatible with standard size bag valve masks and anesthesia circuits. The body of the lower face oxygen mask also provides an access port for inlet and outlet conduits for oxygen and carbon dioxide flow.

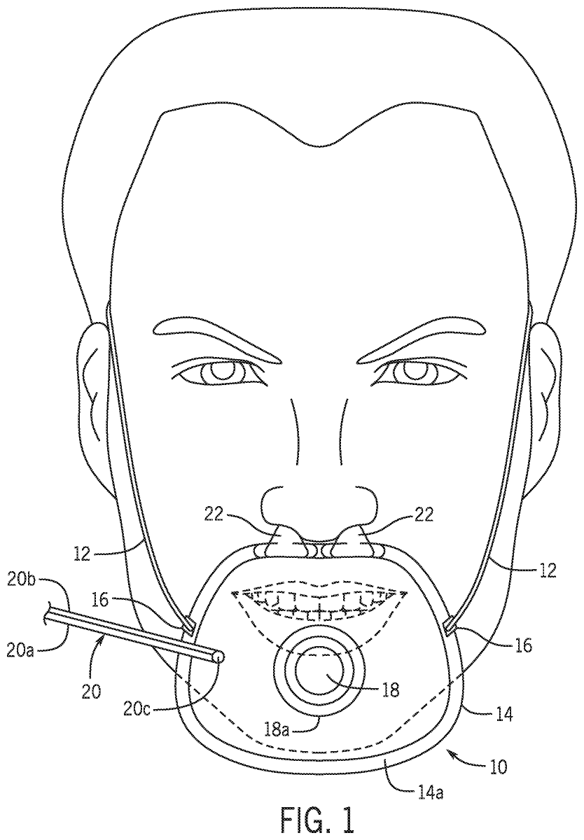

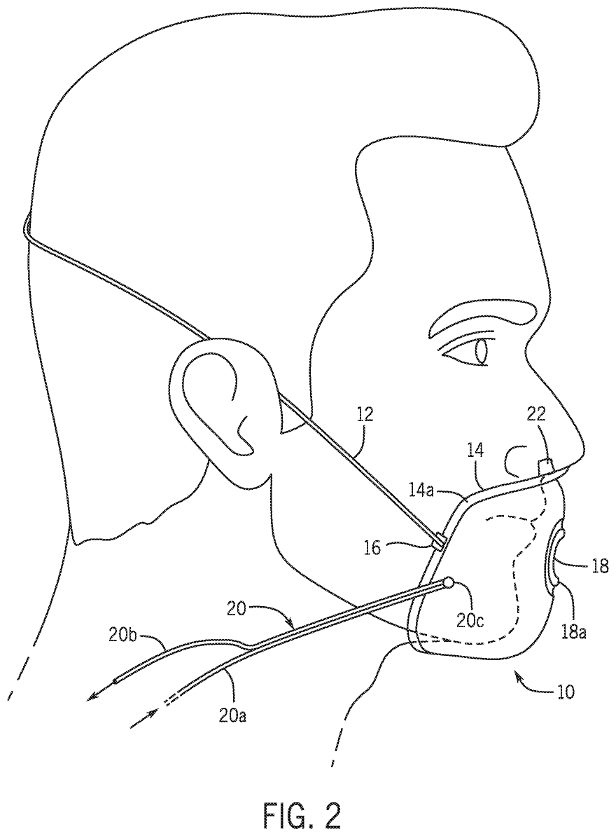

Referring now to FIGS. 1 and 2, the present invention may include a lower face oxygen mask 10 dimensioned and adapted to cover just the chin and mouth of the wearer. The lower face oxygen mask 10 has a body 14 defined by a periphery 14a. Though the figures show an oval design of the body 14, other shapes are contemplated so long as they function as disclosed herein. In certain embodiments, the periphery 14a may be an inflatable balloon cuff. The body 14 may be made of transparent material.

A band 12, most likely elastic, may interconnect two portions of the periphery 14a for securing the lower face oxygen mask 10 to a wearer. At the interface of the band and the periphery 14a may be pull tabs 16 for tightening the lower face oxygen mask 10.

An access aperture 18 may be provided in the body 14 so as to be in line with the mouth of the wearer. In certain embodiments, the access aperture 18 may be 13 mm in diameter so as to be compatible with bag valve mask (“self-inflating bag”) and anesthesia circuits standard size. In certain embodiments an opening periphery 18a may include corrugated ribs.

Along an upper portion of the periphery 14a may be two spaced apart conical nasal prongs 22 to establish a seal with various sized nostrils and prevent mask movement towards eyes. The nasal prongs 22 are fluidly coupled to an inner side of the lower face oxygen mask 10.

An access port 20c may be provided in the body 14. An inlet conduit 20a and an outlet conduit 20b may be fluidly coupled to the access port 20c. The access port 20c may be located slightly inward of the periphery 14a. The inlet and outlet conduits 20a and 20b may be housed in a single tube 20. The inlet conduit 20a is adapted for oxygen delivery and may be five to seven feet in length with a standard sized inverse conical connector used to connect to oxygen flow meters. The outlet conduit 20b being adapted for a carbon dioxide outlet tube.

All the elements work together to complete purpose of the present invention as a superior mask oxygen delivery device. The present invention can be used in routine cases to provide oxygen to the sedated patient. The lower face oxygen mask 10 can also be used in the endoscopy arena to give access for the endoscopist's upper endoscopy, while adding an increased level of safety with the access aperture 18 being compatible with bag valve masks and anesthesia circuits standard size. Furthermore, the lower face oxygen mask 10 provides more comfort and safety by keeping the mask away from the wearer's eyes.

A method of using the present invention may include the following. The lower face oxygen mask 10 disclosed above may be provided. During upper endoscopy, patients can desaturate rapidly by using this design the endoscopist can rapidly remove the scope and oxygen delivery device such as a bag valve mask or anesthesia circuit for delivery of positive pressure ventilation and high flow oxygen. Additionally, the present invention can be used as a general oxygen delivery device whenever oxygen delivery is needed.

It should be understood, of course, that the foregoing relates to exemplary embodiments of the invention and that modifications may be made without departing from the spirit and scope of the invention as set forth in the following claims.

What is claimed is:

1. A lower face oxygen mask, comprising:
a body defined by a periphery;
the periphery dimensioned and adapted to circumscribe a mouth and a chin of a wearer and be below a nose of the wearer; and
two nostril ports extending upwardly from the periphery for fluidly engages nostrils, respectively, of the wearer and a spaced circumscribed by the body.

2. The lower face oxygen mask of claim 1, further comprising:
an access aperture in the body, the access aperture adjacent said mouth.

3. The lower face oxygen mask of claim 2, wherein the access aperture sized to be compatible with bag valve masks and anesthesia circuits.

4. The lower face oxygen mask of claim 3, wherein the access aperture being 13 mm in diameter.

5. The lower face oxygen mask of claim 2, further comprising one or more corrugated ribs defining the access aperture.

6. The lower face oxygen mask of claim 1, further comprising an access port for an inlet conduit and an outlet conduit.

7. The lower face oxygen mask of claim 1, wherein the periphery is an inflatable balloon cuff.

8. A lower face oxygen mask, comprising:
a body defined by a periphery;
the periphery dimensioned and adapted to circumscribe a mouth and a chin of a wearer and be below a nose of the wearer;
two nostril ports extending upwardly from the periphery for fluidly engages nostrils, respectively, of the wearer and a spaced circumscribed by the body;
an access aperture in the body, the access aperture adjacent said mouth, wherein the access aperture sized to be compatible with bag valve masks and anesthesia circuits; and
an access port for an inlet conduit and an outlet conduit.

9. The lower face oxygen mask of claim 8, further comprising one or more corrugated ribs defining the access aperture.

10. The lower face oxygen mask of claim 8, wherein the periphery is an inflatable balloon cuff.
